(19) United States
(12) Patent Application Publication
(10) Pub. No.: US 2012/0302824 A1
(43) Pub. Date: Nov. 29, 2012

(54) Sex partner robot

(76) Inventor: Rexhep Hasimi, Chicago, IL (US)

(21) Appl. No.: 13/065,221

(22) Filed: May 28, 2011

(51) Int. Cl. A61F 5/00

(52) U.S. Cl. 600/38

(57) ABSTRACT

The Robot sex partner will be sold in a metallic box from factory. The robot will have all the human dimensions, average length and placed in horizontal position, locked with a code key.

The metal box has a security code that after open it let the robot get out and this can be activated by human voice.

Then the owner can name the robot and register his name with a code by voice.

Robot after being activated will give its hand to the human partner and it will walk out of the metal box. Owner with his voice will order robot to execute commands like: stand, sit, go to bed, bend over and all kind of movements, also will be able to establish a conversation and have sexual intercourse choosing the female or male personality of the robot

This robot will help lonely people to get reed of depression due to solitude.

My Idea proposes the use of a robot as a sex partner for female or male.

The Sex partner Robot will have perfect human characteristics and will mimic human movements and actions, The robot will have artificial intelligence due to a high performance computer processor installed in his main computer located in the frontal area of his head.

The robot will be able to establish a conversation with his owner and will have a feature that let the owner choose the voice of the robot and changed between female or male voice. The robot will have the ability of flexing his hip , knees and feet and also will have free movements of arms and wrists. A dildo will be placed in the pubic area and this one will have pumping movements similar to a human been and will secrete a lubricant for ease vaginal or anal penetration.

This lubricant will give more comfort to the sexual experience.

The robot will be designed to perform as a male or female partner according to owner requirements.

Also the robot can participate in a group sex relation because it has the ability to perform with two extra dildos in his hands giving pleasure up to three people.

The robot will have and special mechanism in his spinal column that will permit the robot to bend his hips and legs getting very easy on his knees or on his back opening and spreading his legs to facilitate easy vaginal or anal penetration.

There will be a control located in the robot hand that let owner to control speed of pumping movements of dildo from slower to faster pumping.

Also the hand control will et the owner to adjust position of arms and wrist of robot to assume any desirable position on the floor or bed.

The robot will be able to receive voice commands orders from the owner and also the owner will be able to control it with a remote control.

There will be 2 buttons one in each knee to control extension and flexion of legs facilitating a comfortable position during intercourse.

The sex partner robot will be available in adult stores and will come with security features to avoid its use for not authorized personnel like kids or minors.

The robot will be sold in a metallic box with a security code for safe storage and also will have a voice recognition system that will work only with the voice of its owner and as extra security feature the robot will be able to recognize the face of the owner due to an special face recognition system that will store pictures of the owner and will compare it before its use.

The robot can storage multiple pictures and information for many users.

After opening the box the robot will walk out and will say hello then you will be able to set personal settings for its use.

The robot will be able to lie down on floor, bed or sofa and will establish a conversation will owner, also the robot will perform suction movements with his mouth to perform oral sex to female or male partner.

Robot will be able to bend over or crawl in direction of his owner giving more emotion to sexual relation.

This feature will give the robot an special touch or realistic companionship helping people to get rid of depression and solitude.

### DESCRIPTION FOR PICTURES

PICTURE No 1 and 2. Show the left side view of the model robot in standing position with a dildo in pubic area.
A: HEAD
B: GLASS
C: MONITOR
D: RIGHT HAND
E: RIGHT FINGERS
F: DILDO
H: HARNESS
I: LEFT HAND
J: BUTTOM TO STRETCH LEFT HAND
K: LEFT FINGERS
L: LEFT FOOD

PICTURE No 3. Shows a frontal view of robot with dildo supported for a harness.

PICTURE No 4. Shows the back of robot with harness.

PICPICTURE No 5. Shows a right view of the robot in standing position with a dildo in pubic area.

PICTURE No 6 and 7. Show the left view of the robot in standing position with a dildo in his hand and one in his pubic area.

The invention claimed is:

1. A method of artificial companionship to help lonely people to have a better sexual life without a human companion.

2. The method of claim 2, where the cost of production affecting prices will be reduce when robot gets mass production.

3. The method of claim 3, where the robot will posses artificial intelligence due to a super fast computer processor located in the front of the head of robot.

4. The method of claim 4, where robot will be able to fallow verbal orders and human movements.

5. The method of claim 5, where the robot will mimic human movements giving him a realistic human appearance.
